(19) United States
(12) Patent Application Publication
(10) Pub. No.: US 2011/0165136 A1
(43) Pub. Date: Jul. 7, 2011

(54) Novel Herbal Composition for Treatment of Psoriasis and Other Skin Disorders

(75) Inventors: Uddagiri Venkanna Babu, Bangalore (IN); Bhaidyanath Mishra, Bangalore (IN)

(73) Assignee: HIMALAYA GLOBAL HOLDINGS LTD., Dubai (IN)

(21) Appl. No.: 12/981,742

(22) Filed: Dec. 30, 2010

(30) Foreign Application Priority Data
Jan. 1, 2010 (IN) 5/DEL/2010

(51) Int. Cl. A61K 36/31; A61P 17/06; A61K 38/43

(52) U.S. Cl. 424/94.1; 424/755

(57) ABSTRACT

The invention relates to a herbal composition for the treatment of psoriasis and other skin disorders. The herbal composition comprises the extracts of Psoralea corylifolia, Luffa acutangula, Rubia cordifolia, Boswellia serrata and Brassica nigra and oils of Azhadirachta indica, Linum usitatissum, Vitis vinifera, Brassica nigra, and Pongamia seed. The present invention further relates to cosmeceutical compositions comprising the said active agents, methods for preparing the composition and using the same in Psoriasis and other skin disorders in human beings. The methods of making the plant extract, methods for obtaining the topical preparation are disclosed.

### FIELD OF THE INVENTION

The present invention relates to a herbal composition useful in treatment of psoriasis. More particularly, the invention relates to a composition comprising extracts of Psoralia corylifolia seeds and/or Luffa acutangula seeds and/or Rubia cordifolia roots and/or Boswellia serrata gum resins and/or Brassica nigra seeds and oils of Azadirachta indica seeds and/or Linum usitatissimum seeds and/or Vitis vinifera seeds and/or Brassica nigra seeds and/or Pongamia seeds and a cosmeceutically acceptable carrier, methods of obtaining the same, cosmeceutical formulations thereof and methods of using the composition for treatment of psoriasis, a chronic and non-contagious auto-immune disease that affects the skin and joints and other related skin disorders in humans.

### BACKGROUND OF THE INVENTION

Psoriasis is a chronic, non-contagious autoimmune disease that affects the skin and joints. It commonly causes red, scaly patches on the skin. The scaly patches caused by psoriasis are called psoriatic plaques. The skin accumulates rapidly at these sites and becomes silvery-white in appearance. Plaques occur frequently on the skin of the elbows and knees but can affect any area including the scalp and genitals. In contrast to eczema, psoriasis is more likely to be found on the extensor aspect of the joints.

This disorder is a chronic recurring condition that varies in severity from minor localized patches to complete body coverage. Generally fingernails and toenails are affected by the infection (psoriatic nail dystrophy) and can be seen as an isolated finding. Psoriasis can also cause inflammation of the joints, which is known as psoriatic arthritis. Ten to fifteen percent of the total people with psoriasis have psoriatic arthritis.

Psoriasis is the most prevalent autoimmune disease in the U.S. According to the National Institutes of Health (NIH), as many as 7.5 million Americans—approximately 2.2 percent of the population—have psoriasis. 125 million people worldwide—2 to 3 percent of the total population has psoriasis. Studies have shown that between 10 and 30 percent of people with psoriasis also develop psoriatic arthritis. Psoriasis prevalence in African Americans is 1.3 percent compared to 2.5 percent of Caucasians.

The direct and indirect health care costs of psoriasis for patients are calculated at $11.25 billion annually with work loss accounting for 40 percent of the cost burden. Approximately 60 percent of psoriasis patients missed an average of 26 days of work a year due to their illness.

The treatment for psoriasis is mainly topical application, phototherapy and systemic administration of antibiotics, as pills or injections in case of severe psoriasis and psoriatic arthritis. There are many medications and synthetic drugs available for psoriasis. FDA has approved three new treatment options for psoriasis patients such as Taclonex Scalp, a new topical ointment for treating scalp psoriasis, Xtrac velocity excimer laser system, which emits a high-intensity beam of ultraviolet light that can treat moderate to severe psoriasis and a drug molecule adalimumab approved to treat moderate to severe psoriasis. Adalimumab had already been approved to treat psoriatic arthritis. Over times, psoriasis can become resistant to a specific therapy. Treatments may be periodically changed to prevent resistance developing (tachyphylaxis) and to reduce the chance of occurring adverse reactions.

It is therefore very important to develop safe and effective medications for psoriasis and other related skin disorders from natural sources. The present invention involves the identification of herbal extracts and oils in a unique combination that can be effectively formulated as topical application for treatment of psoriasis without any side affects.

### OBJECT OF THE INVENTION

It is an object of the present invention to provide a herbal formulation for the treatment of psoriasis and other related skin disorders.

Another object of the invention is to obviate the disadvantages and side effects of the available treatment methods and compositions.

Still another object of this invention is to prepare a cosmeceutical composition from the said herbal extracts and methods of preparing the composition for human skin disorders.

Yet another object of this invention is to provide a method of preparation of a herbal formulation for the treatment of psoriasis and other related skin disorders.

### SUMMARY OF THE INVENTION

The present invention relates to a herbal composition useful in treatment of psoriasis. More particularly, the invention relates to a composition comprising extracts of Psoralia corylifolia seeds and/or Luffa acutangula seeds and/or Rubia cordifolia roots and/or Boswellia serrata gum resins and/or Brassica nigra seeds and oils of Azadirachta indica seeds and/or Linum usitatissimum seeds and/or Vitis vinifera seeds and/or Brassica nigra seeds and/or Pongamia seeds and a cosmeceutically acceptable carrier, methods of obtaining the same, cosmeceutical formulations thereof and methods of using the composition for treatment of psoriasis, a chronic and non-contagious auto-immune disease that affects the skin and joints and other related skin disorders in humans.

According to the present invention, a herbal formulation, for treatment of psoriasis, a chronic and non-contagious auto-immune disease that affects the skin and joints and other related skin disorders in humans is disclosed. The inventive herbal formulation comprises extracts of Psoralia corylifolia seed extract, Luffa acutangula seed extract, Rubia cordifolia root extract, Brassica nigra seed extract, Boswellia serrata gum resin, Azhadirachta indica seed oil, Linum usitatissimum seed oil, Vitis vinifera seed oil, Brassica nigra seed oil and/or Pongamia seeds and a cosmeceutically acceptable carrier. The herbal formulation according to the present invention is useful for treating psoriasis and other skin disorders.

### BRIEF DESCRIPTION OF DRAWINGS

FIGS. 1-4 depict the evaluation chart of topical antipsoriatic activity of AP series by Mouse tail test.

### EMBODIMENT OF THE INVENTION

In a preferred embodiment of the invention a herbal composition having anti-psoriatic activity comprises effective amounts of extracts of Psoralia corylifolia, Luffa acutangula, Rubia cordifolia, Boswellia serrata, Brassica nigra, oils of Azadirachta indica, Linum usitatissimum, Vitis vinifera, Brassica nigra, and suitable cosmeceutically acceptable carriers and additives wherein the said composition is in lotion or cream formulation.

In another embodiment the herbal extracts are obtained either by separate extraction or from the blend of Psoralia corylifolia, Luffa acutangula, Rubia cordifolia, Boswellia serrata, Brassica nigra, and oils of Azadirachta indica, Linum usitatissimum, Vitis vinifera, Brassica nigra parts.

In yet another embodiment of the invention the percent weight by weight composition comprises 0.5-2% Psoralia corylifolia, 0.1-1% Luffa acutangula, 0.05-1% Rubia cordifolia, 0.05-1% Boswellia serrata, 1-3% Brassica nigra extract, 0.5-4% Azadirachta indica, 0.01-1% Linum usitatissimum, 0.2-2% Vitis vinifera, 0.1-2% Brassica nigra oil and cosmeceutically acceptable carriers.

In another preferred embodiment, the herbal composition extracts are obtained from the specific parts of the herbs preferably seeds of Psoralia corylifolia, seeds of Luffa acutangula, roots of Rubia cordifolia, gum resins of Boswellia serrata, seeds of Brassica nigra, and the oils are obtained from the specific parts of herbs preferably seeds of Azadirachta indica, seeds of Linum usitatissimum, seeds of Vitis vinifera, seeds of Brassica nigra.

In another embodiment, the suitable cosmeceutical carrier in the composition is selected from the group of Xanthum gum, Aloe vera water, Glycerine, Carrageenan gum, Isopropyl Myristate, or any other carrier known in the art, or mixtures or combinations thereof.

In another preferable embodiment, a herbal lotion having anti-psoriatic activity comprises of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 1.5-3% w/w alcoholic extract of Brassica nigra, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

In yet another preferred embodiment a herbal lotion having anti-psoriatic activity comprises of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 0.5-2% w/w Psoralea corylifolia seed extract, 0.2-1% w/w Luffa acutangula seed extract, 0.05-0.8% w/w Rubia cordifolia root extract, 0.05-0.8% w/w Boswellia serrata gum resin extract, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

In another embodiment the herbal extracts are obtained by method selected from the group of percolation method, hot-soxlation method or super-critical-fluid method. In still another embodiment a herbal lotion having anti-psoriatic activity comprises of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 1.5-3% w/w herbal blend extract, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

In still another embodiment of the invention the herbal blend comprises of the seeds of Psoralia corylifoia, seeds of Luffa acutangula, roots of Rubia cordifolia and gum resins of Boswellia serrata in the ratio range of 45:20:15:20 to 50:25:10:15.

In another embodiment the herbal blend is prepared by percolation method or hot-soxlation method of extraction.

In yet another preferred embodiment of the invention a herbal cream having anti-psoriatic activity comprises of 3-5% w/w cetearyl Olivate and sorbitan olivate, 10-16% w/w sesame oil, 10-20% w/w Bees wax, 2-8% w/w isopropyl myristate, 0.1-1% w/w sorbic acid, 0.2-1.5% w/w Brassica nigra seed oil, 0.2-1.5% w/w Vitis vinifera seed oil, 0.1-1% w/w Linium usiatissimum seed oil, 0.5-2% w/w Pongamia seed oil, 5-15% w/w glycerine, 0.1-1% w/w Xanthum gum, 0.05-1% carrageenan gum, 1-5% w/w coco glucoside, 1-5% w/w Brassica nigra extract, 0.05-1% w/w sodium benzoate, 0.05-1% w/w potassium sorbate, 0.05-1% w/w Vitamin E acetate, 0.05-2% w/w perfume and 35-50% w/w purified water.

In still another embodiment of the proposed invention the method of preparation of a herbal lotion having anti-psoriatic activity comprises the following steps of:
a. Weighing the oil phase ingredients separately and heating the same in sterilized vessels up to a temperature ranging from 75-85° C.;
b. Dispersing xanthum gum in 10% Aloe vera water;
c. Adding glycerin-water and mixing the same with the alcoholic extract of Brassica nigra;
d. Heating the vessel containing water phase ingredients obtained in steps b. and c. to a temperature ranging from 75-85° C.;
e. Mixing the oil phase obtained in step a. with water phase obtained in step d. at temperature ranging from 75-85° C. and stirring the same properly; and
f. Mixing the preservatives in the phase obtained in step e. at temperature range less than 40° C. and stirring the same properly.

In still another preferred embodiment of the proposed invention the method of preparation of a herbal cream having anti-psoriatic activity comprises the following steps of:
a. Heating Cetearyl Olivate & Sorbitan Olivate, sesame oil, Bees wax, isopropyl myristate, Sorbic acid and pongamia oil to a temperature ranging from 75-85° C. (Phase A);
b. Dispersing Xanthan gum and carrageenan gum in glycerine, adding this dispersion to purified water in main vessel, adding coco-glucoside in the main vessel, dissolving sodium benzoate and potassium sorbate in small quantity of water and adding the same to the main vessel, followed by adding Brassica nigra extract in the main vessel, heating the mixture thus obtained to a temperature ranging from 75-85° C. (Phase B);
c. Adding phase A to Phase B at a temperature ranging from 75-85° C. under homogenizer for 15-25 minutes;
d. Cooling the homogenized mixture obtained in step c. with normal water;
e. Adding Vitamin E acetate and perfume at a temperature ranging from 40-50° C.;
f. Mixing the content obtained in step e. for 10-20 minutes.

### DETAILED DESCRIPTION OF THE INVENTION

The present invention relates to a herbal composition useful in treatment of psoriasis. More particularly, the invention relates to a composition comprising extracts of Psoralia corylifolia seeds and/or Luffa acutangula seeds and/or Rubia cordifolia roots and/or Boswellia serrata gum resins and/or Brassica nigra seeds and oils of Azadirachta indica seeds and/or Linum usitatissimum seeds and/or Vitis vinifera seeds and/or Brassica nigra seeds and/or Pongamia seeds and a cosmeceutically acceptable carrier, methods of obtaining the same, cosmeceutical formulations thereof and methods of using the composition for treatment of psoriasis, a chronic and non-contagious auto-immune disease that affects the skin and joints and other related skin disorders in humans.

According to the present invention, a herbal formulation, for treatment of psoriasis, a chronic and non-contagious auto-immune disease that affects the skin and joints and other related skin disorders in humans is disclosed. The inventive herbal formulation comprises extracts of Psoralia corylifolia seed extract, Luffa acutangula seed extract, Rubia cordifolia root extract, Brassica nigra seed extract, Boswellia serrata gum resin, Azhadirachta indica seed oil, Linum usitatissimum seed oil, Vitis vinifera seed oil, Brassica nigra seed oil, Pongamia seed oil and a cosmeceutically acceptable carrier. The herbal formulation according to the present invention is useful for treating psoriasis and other skin disorders.

The present invention involves the selection and identification of the herbs and obtaining the extracts by subjecting the same to different extraction techniques including conventional solvent extraction and super critical fluid extraction. The bioassay guided fractionation of the extract or combination thereof to identify the active markers or active fraction and to develop effective and safe composition for the use in human beings for treatment of all types of psoriasis and other related skin disorders in humans.

The following methodology, examples, and studies of the composition follow the teachings of and illustrate the present invention.

### Example-1

Preparation of Extract from Psoralea corylifolia by Percolation Method:

The shade dried material of seeds of Psoralia corylifolia was pulverized to coarse powder and about 10 Kg each of powdered material is placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness by rotatory evaporator or on steam bath at optimum temperature and under reduced pressure.

### Example-2

Preparation of Extract from Psoralia corylifolia by Hot-Soxlation Method:

The coarse powdered material of seeds of Psoralea corylifolia was subjected to hot-soxlation by placing 10 Kg of material in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness by rotatory evaporator or on steam bath at optimum temperature.

All extracts such as n-hexane extract (APPC-1), acetone extract (APPC-2), ethyl alcohol extract (APPC-3), methanol extract (APPC-4), ethyl alcohol and water (1:1) extract (APPC-5), methanol and water (1:1) extract (APPC-6) and water extract (APPC-7) prepared from the seeds of Psoralea corylifolia by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-3

Preparation of Extract from Psoralea corylifolia by Super Critical Fluid Extraction

The shade dried material of seeds of Psoralea corylifolia was pulverized to coarse powder and about 100 Kg of powdered material was placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-4

Preparation of Extract from Luffa acutangula by Percolation Method:

The shade dried material of seeds of Luffa acutangula was pulverized to coarse powder and about 10 Kg each of powdered material was placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness by rotatory evaporator or on steam bath at optimum temperature and under reduced pressure.

### Example-5

Preparation of Extract from Luffa acutangula by Hot-Soxlation Method:

The coarse powdered material of seeds of Luffa acutangula was subjected to hot-soxlation by placing 10 Kg of material in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness on rotatory evaporator or on steam bath at optimum temperature.

All extracts such as n-hexane extract (APLA-1), acetone extract (APLA-2), ethyl alcohol extract (APLA-3), methanol extract (APLA-4), ethyl alcohol and water (1:1) extract (APLA-5), methanol and water (1:1) extract (APLA-6) and water extract (APLA-7) prepared from the seeds of Luffa acutangula by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-6

Preparation of Extract from Luffa acutangula by Super Critical Fluid Extraction

The shade dried material of seeds of Luffa acutangula was pulverized to coarse powder and about 100 Kg of powdered material was placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-7

Preparation of Extract from Rubia cordifolia by Percolation Method:

The shade dried material of roots of Rubia cordifolia was pulverized to coarse powder and about 10 Kg each of powdered material was placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness on by rotatory evaporator or on steam bath at optimum temperature and under reduced pressure.

### Example-8

Preparation of Extract from Rubia cordifolia by Hot-Soxlation Method:

The coarse powdered material of roots of Rubia cordifolia was subjected to hot-soxlation by placing 100 Kg of material in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness on by rotatory evaporator or on steam bath at optimum temperature.

All extracts such as n-hexane extract (APRC-1), acetone extract (APRC-2), ethyl alcohol extract (APRC-3), methanol extract (APRC-4), ethyl alcohol and water (1:1) extract (APRC-5), methanol and water (1:1) extract (APRC-6) and water extract (APRC-7) prepared from the roots of Rubia cordifolia by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-9

Preparation of Extract from Rubia cordifolia by Super Critical Fluid Extraction

The shade dried material of roots of Rubia cordifolia was pulverized to coarse powder and about 100 Kg of powdered material placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-10

Preparation of Extract from Boswellia serrata by Percolation Method:

The shade dried material of gum resins of Boswellia serrata was pulverized to coarse powder and about 10 Kg each of powdered material placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness on rotatory evaporator or on steam bath at optimum temperature and under reduced pressure.

### Example-11

Preparation of Extract from Boswellia serrata by Hot-Soxlation Method:

The coarse powdered material of gum resins of Boswellia serrata was subjected to hot-soxlation by placing 10 Kg of material in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness on by rotatory evaporator or on steam bath at optimum temperature.

All extracts such as ethyl acetate extract (ASBE-1), acetone extract (ASBE-2), ethyl alcohol extract (ASBE-3), methanol extract (ASBE-4), ethyl alcohol and water (1:1) extract (ASBE-5), methanol and water (1:1) extract (ASBE-6) and water extract (ASBE-7) prepared from the gum resins of Boswellia serrata by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-12

Preparation of Extract from Boswellia serrata by Super Critical Fluid Extraction

The shade dried material of gum resins of Boswellia serrata was pulverized to coarse powder and about 100 Kg of powdered material placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-13

Preparation of Extract from Brassica nigra by Percolation Method:

The shade dried material of seeds of Brassica nigra was pulverized to coarse powder and about 10 Kg each of powdered material placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness by on rotatory evaporator or on steam bath at optimum temperature and under reduced pressure.

### Example-14

Preparation of Extract from Brassica nigra by Hot-Soxlation Method:

The coarse powdered material of seeds of Brassica nigra was subjected to hot-soxlation by placing 10 Kg of material in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness on by rotatory evaporator or on steam bath at optimum temperature.

All extracts such as n-hexane extract (APBN-1), acetone extract (APBN-2), ethyl alcohol extract (APBN-3), methanol extract (APBN-4), ethyl alcohol and water (1:1) extract (APBN-5), methanol and water (1:1) extract (APBN-6) and water extract (APBN-7) prepared from the seeds of Brassica nigra by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-15

Preparation of Extract from Brassica nigra by Super Critical Fluid Extraction

The shade dried material of seeds of Brassica nigra was pulverized to coarse powder and about 100 Kg of powdered material placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-16

Preparation of Extract from Herbal Blend by Percolation Method

The shade dried material of herbal blend from the seeds of Psoralia corylifoia, seeds of Luffa acutangula, roots of Rubia cordifolia and gum resins of Boswellia serrata in the ratio of 50:25:10:15 was pulverized to coarse powder and about 10 Kg each of powdered herbal blend placed in different percolators and extracted with n-hexane, acetone, ethyl alcohol, methanol, ethyl alcohol and water (1:1), methanol and water (1:1) and water at room temperature for 24 h to 48 h., then plant extracts were filtered and concentrated to dryness on rotatory evaporator or on steam bath at optimum temperature and under reduced pressure.

### Example-17

Preparation of Extract from Herbal Blend by Hot-Soxlation Method:

The coarse powdered material of herbal blend from the seeds of Psoralia corylifoia, seeds of Luffa acutangula, roots of Rubia cordifolia and gum resins of Boswellia serrata in the ratio of 50:25:10:15 was subjected to hot-soxlation by placing 10 Kg of herbal blend in each soxlator using solvents n-hexane, acetone, ethyl alcohol, methanol, ethyl alcohol and water (1:1), methanol and water (1:1) and water at refluxing temperature of each solvent and recycled the process until extraction is completed, then plant extracts were filtered and concentrated to dryness on rotatory evaporator or on steam bath at optimum temperature.

All extracts such as n-hexane extract (AP(A)-1), acetone extract (AP(A)-2), ethyl alcohol extract (AP(A)-3), methanol extract (AP(A)-4), ethyl alcohol and water (1:1) extract (AP(A)-5), methanol and water (1:1) extract (AP(A)-6) and water extract (AP(A)-7) prepared from herbal blend from the seeds of Psoralia corylifoia, seeds of Luffa acutangula, roots of Rubia cordifolia and gum resins of Boswellia serrata in the ratio of 50:25:10:15 by percolation method or hot-soxlation method were subjected to HPTLC (High Performance Thin Layer Chromatography) and HPLC (High performance Liquid chromatography) in various mobile phases combinations on precoated TLC plates (Merck) and ODS column for qualitative and quantitative estimation of marker compounds and active principles.

### Example-18

Preparation of Extract from Herbal Blend by Super Critical Fluid Extraction

The shade dried material of herbal blend from the seeds of Psoralia corylifoia, seeds of Luffa acutangula, roots of Rubia cordifolia and gum resins of Boswellia serrata in the ratio of 50:25:10:15 was pulverized to coarse powder and about 100 Kg of powdered material was placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-19

Preparation of Extract from Azhadirachta indica by Super Critical Fluid Extraction

The shade dried material of seeds of Azhadirachta indica was pulverized to coarse powder and about 100 Kg of powdered material was placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in the highest pure form.

### Example-20

### Preparation of Extract from Linum usitatissum by Super Critical Fluid Extraction

The shade dried material of seeds of Linum usitatissum was pulverized to coarse powder and about 100 Kg of powdered material was placed in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in highest pure form.

### Example-21

Preparation of extract from Vitis vinifera by Super Critical Fluid Extraction

The shade dried material of seeds of Vitis vinifera was pulverized to coarse powder and about 100 Kg of powdered material placed was in a SCF extractor at the temperature of 40-50° C. at high pressure of 300-350 bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in highest pure form.

### Example-22

Preparation of Extract from Brassica nigra by Super Critical Fluid Extraction

The shade dried material of seeds of Brassica nigra was pulverized to coarse powder and about 100 Kg of powdered material was placed in a SCF extractor at the temperature of 40-50° C. at high pressure of bar using carbon dioxide as super critical fluid for extraction upto 4 to 6 hours and then the extract was collected in the collection vessel and evaporated at room temperature to remove any further residues of carbon dioxide. The extract thus obtained was free from any solvent residues and is in highest pure form.

### Example-23

Preparation of Oils from Azhadirachta indica by Cold Press Method

The shade dried material of seeds of Azhadirachta indica was subjected to cold press oil extraction process at room temperature and the final oil obtained from the crushing of the seeds was filtered through muslin cloth and stored in an air tight containers.

### Example-24

Preparation of Oils from Linum Usitatissum by Cold Press Method

The shade dried material of seeds of Linum usitatissum was subjected to cold press oil extraction process at room temperature and the final oil obtained from the crushing of the seeds was filtered through muslin cloth and stored in an air tight containers.

### Example-25

Preparation of Oils from Vitis vinifera by Cold Press Method

The shade dried material of seeds of Vitis vinifera was subjected to cold press oil extraction process at room temperature and the final oil obtained from the crushing of the seeds was filtered through muslin cloth and stored in an air tight containers.

### Example-26

Preparation of Oils from Brassica Nigra by Cold Press Method

The shade dried material of seeds of Brassica nigra was subjected to cold press oil extraction process at room temperature and the final oil obtained from the crushing of the seeds was filtered through muslin cloth and stored in an air tight containers.

Preparation of Oils from Pongamia by Cold Press Method

The shade dried material of seeds of Pongamia was subjected to cold press oil extraction process at room temperature and the final oil obtained from the crushing of seeds was filtered through muslin cloth and stored in air tight containers.

### Example-27

### Composition of Antipsoriasis Lotion (Formula-1) Batch Size: 500 g

TABLE 1

| SL. NO. | INGREDIENTS | PERCENTAGE | QUANTITY (g) |
|---|---|---|---|
| 1. | Azhadirachta indica | 1.05 | 5.25 |
| | Seed Oil | | |
| 2. | Linum usitatissimum | 0.45 | 2.25 |
| | Seed Oil | | |
| 3. | Vitis vinifera Seed Oil | 0.75 | 3.75 |
| 4. | Brassica nigra | 0.75 | 3.75 |
| | Seed Oil | | |
| 5. | Brassica nigra | 2.0 | 10 |
| | Alcoholic extract | | |
| 6. | Sunflower Oil | 4.0 | 20 |
| 7. | Almond Oil | 4.0 | 20 |
| 8. | Cocoa butter | 0.2 | 1.0 |
| 9. | Olivem 1000 | 3.0 | 15.0 |
| 10. | Aloe vera Water | 78.95 | 394.75 |
| 11. | Xanthum gum | 0.3 | 1.5 |
| 12. | Glycerin | 3.0 | 1.5 |
| 13. | Biovert Enzyme | 0.05 | 0.25 |
| 14. | Sodium benzoate | 0.5 | 2.5 |

### Example-28

### Brief Manufacturing Procedure of Antipsoriatic Lotion

1. Weigh the oil phase ingredients in separate separately and heat in a sterilized vessels and heat up to 80° C.
2. Disperse xanthum gum in 10% Aloe vera water was dispersed.
3. Add glycerin-water and mixed with the Brassica nigra extract.
4. Heat the vessel containing above water phase ingredients to 80° C.
5. Mix oil phase to in water phase at @ 80° C. and stir well.
6. At <40° C. mix the preservatives and stir well. Mix the preservatives and stir well at less than 40° C.

### Example-29

### Composition of Antipsoriasis Lotion (Formula-2) Batch Size: 500 g

TABLE 2

| SL. NO. | INGREDIENTS | PERCENTAGE | QUANTITY (g) |
|---|---|---|---|
| 1 | Azhadirachta indica | 1.05 | 5.25 |
| | Seed Oil | | |
| 2 | Linum usitatissimum | 0.45 | 2.25 |
| | Seed Oil | | |
| 3 | Vitis vinifera Seed Oil | 0.75 | 3.75 |
| 4 | Brassica nigra | 0.75 | 3.75 |
| | Seed Oil | | |
| 5 | Psoralea corylifolia | 1.0 | 5 |
| | Seed extract | | |
| 6 | Luffa acutangula | 0.5 | 2.5 |
| | Seed extract | | |
| 7 | Rubia cordifolia | 0.2 | 1.0 |
| | Root extract | | |
| 8 | Boswellia serrata | 0.3 | 1.5 |
| | Gum Resin extract | | |
| 9 | Sunflower Oil | 4.0 | 20 |
| 10 | Almond Oil | 4.0 | 20 |
| 11 | Cocoa butter | 0.2 | 1.0 |
| 12 | Olivem 1000 | 3.0 | 15.0 |
| 13 | Aloe vera Water | 78.95 | 394.75 |
| 14 | Xanthum gum | 0.3 | 1.5 |
| 15 | Glycerin | 3.0 | 1.5 |
| 16 | Biovert Enzyme | 0.05 | 0.25 |
| 17 | Sodium benzoate | 0.5 | 2.5 |

### Example-30

### Brief Manufacturing Procedure of Antipsoriasis Lotion

1. Weigh the oil phase ingredients in separately and heat in a sterilized vessels at temperature up to 80° C.
2. Disperse xanthum gum in 10% Aloe vera water.
3. Add glycerin-water mixed with extract.
4. Heat the vessel containing above water phase ingredients to 80° C.
5. Mix oil phase to water phase at 80° C. and stir well.
6. Mix the preservatives and stir well at less than 40° C.

### Brief Manufacturing Procedure of Antipsoriasis Cream

1. Heated Cetearyl Olivate & Sorbitan Olivate, sesame oil, Bees wax, isopropyl myristate, Sorbic acid and pongamia oil to 80° C. (Phase A)
2. Disperse Xanthan gum and carrageenan gum in glycerine. Added this dispersion to purified water in main vessel. Added coco-glucoside in the main vessel. Dissolved sodium benzoate and potassium sorbate in small quantity of water and add to the main vessel. Added Brassica Nigra Extract in the main vessel. Heated to 80° C. (Phase B)
3. Added phase A to Phase B at 80° C. under homogenizer for 20 min.
4. Started cooling with normal water.
5. Added Vitamin E acetate and perfume at 45° C.
6. Mixed for 15 min.

### Example-31

### Composition of Antipsoriasis Lotion (Formula-3) Batch Size: 500 g

TABLE 3

| SL. | | | QUANTITY |
|---|---|---|---|
| NO. | INGREDIENTS | PERCENTAGE | (g) |
| 1. | Azhadirachta indica Seed Oil | 1.05 | 5.25 |
| 2. | Linum usitatissimum Seed Oil | 0.45 | 2.25 |
| 3. | Vitis vinifera Seed Oil | 0.75 | 3.75 |
| 4. | Brassica nigra Seed Oil | 0.75 | 3.75 |
| 5. | Herbal Blend Extract (AP-A) | 2.0 | 10 |
| 6. | Sunflower Oil | 4.0 | 20 |
| 7. | Almond Oil | 4.0 | 20 |
| 8. | Cocoa butter | 0.2 | 1.0 |
| 9. | Olivem 1000 | 3.0 | 15.0 |
| 10. | Aloe vera Water | 78.95 | 394.75 |
| 11. | Xanthum gum | 0.3 | 1.5 |
| 12. | Glycerin | 3.0 | 1.5 |
| 13. | Biovert Enzyme | 0.05 | 0.25 |
| 14. | Sodium benzoate | 0.5 | 2.5 |

### Example-32

### Composition of Antipsoriasis Cream (Formula-4) Batch Size: 500 g

TABLE 4

| SL. | | | QUANTITY |
|---|---|---|---|
| NO. | INGREDIENTS | PERCENTAGE | (g) |
| 1 | Cetearyl Olivate & | 4.00 | 20.00 |
| | Sorbitan Olivate | | |
| 2 | Sesame Oil | 14.00 | 70.00 |
| 3 | Bees wax | 15.00 | 75.00 |
| 4 | Isopropyl Myristate | 5.00 | 25.00 |
| 5 | Sorbic acid | 0.30 | 1.50 |
| 6 | Brassica nigra seed oil | 0.75 | 3.75 |
| 7 | Vitis vinifera seed oil | 0.75 | 3.75 |
| 8 | Linium usiatissum seed oil | 0.45 | 2.25 |
| 9 | Pongamia seed oil | 1.05 | 5.25 |
| 10 | Glycerin | 10.00 | 50.00 |
| 11 | Xanthan Gum | 0.40 | 2.00 |
| 12 | Carrageenan gum | 0.30 | 1.50 |
| 13 | Coco Glucoside | 2.00 | 10.00 |
| 14 | Brassica nigra extract | 2.00 | 10.00 |
| 15 | Sodium Benzoate | 0.20 | 2.5 |
| 16 | Potassium Sorbate | 0.20 | 2.5 |
| 17 | Vitamin E acetate | 0.20 | 2.5 |
| 18 | Perfume | 0.50 | 2.5 |
| 19 | Purified Water | 42.90 | 214.5 |
| | Total quantity | 100 | 500 |

### Pharmacological Screening of the Extracts and Oils for Topical Anti-Psoriatic Activity by Mouse Tail Test

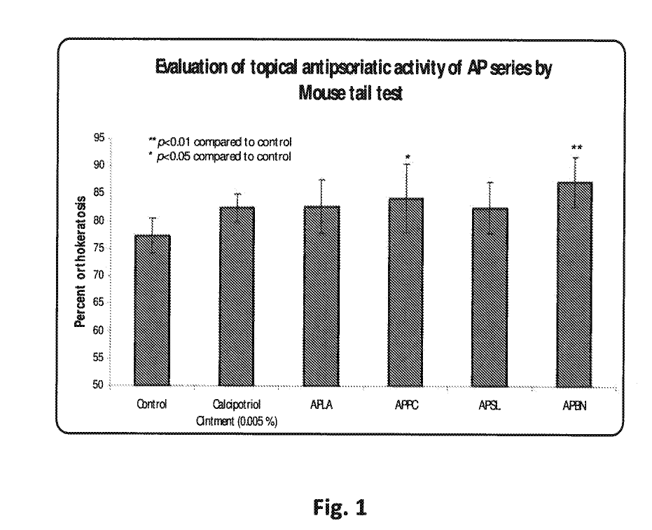

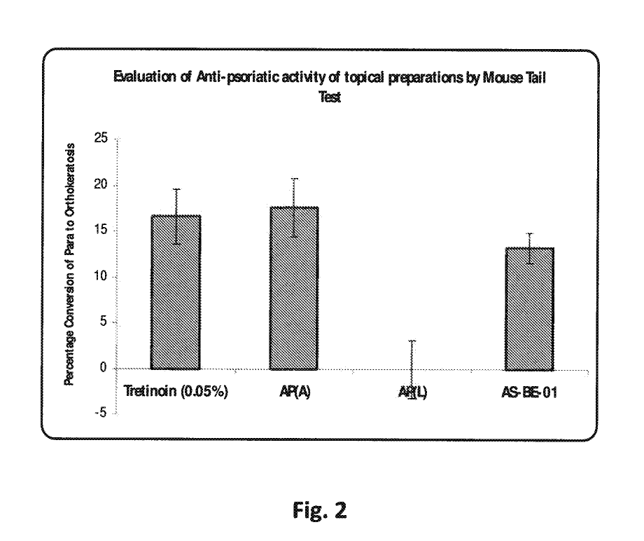

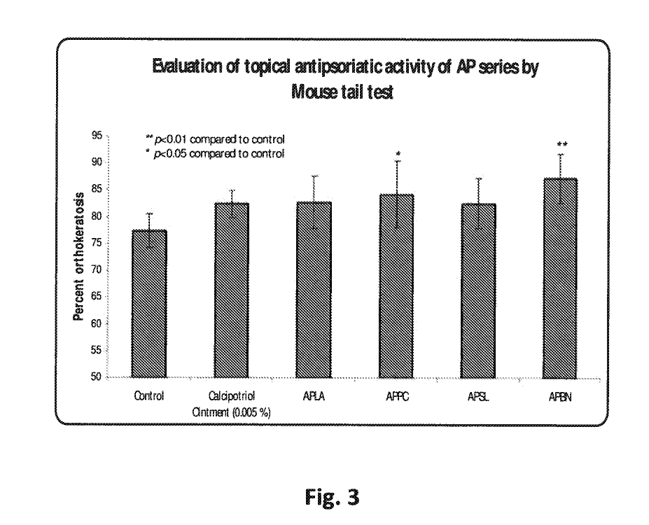

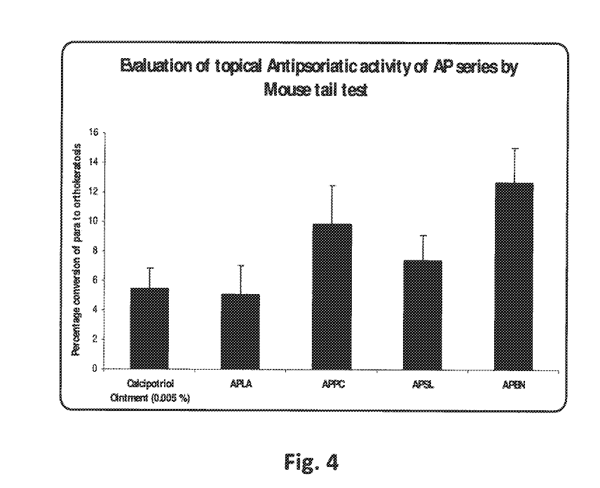

The composite extracts, oils blends and individual alcohol extracts were subjected to screening for evaluation of Anti-psoriatic activity by Mouse tail test and the results are summarized as FIG. 1-4.

### Clinical Study to Evaluate Safety and Efficacy of Antipsoriasis Cream

An open clinical study was conducted to evaluate the efficacy and safety of Antipsoriasis cream in patients suffering from mild to moderate psoriasis. The study was conducted at Zawar's Dermatology Clinic, 21, Shreeram Sankul, Opposite Hotel Panchavati, Vakilwadi, Nashik, India

Ten patients of both sexes, aged more than 18 years, who were clinically diagnosed as suffering from mild to moderate Psoriasis with erythema (redness), induration (thickness) and desquamation dry silvery scaling. Informed consent were obtained from the patients who were willing to take part in the study and enrolled on the study. Patients with any systemic illness or those patients who are not willing to follow the requirements of the study were not included in the study. The subjects who have qualified the screening and willing to participate in the study were called for the study, All the subjects were given antipsoriasis cream to be applied twice daily on the affected areas for a period of 3 months.

The evaluation for the clinical parameters was done at the beginning and at the end of every month for 3 months of the study. The clinical parameters of Psoriasis evaluated are PASI score, erythema (redness), induration (thickness) and desquamation (scaling) Auspitz sign. Severity parameters were measured on a scale of 0 to 4, from none to maximum.

Psoriasis Area Severity Index (PASI) is the widely used tool for the measurement of severity of psoriasis. PASI combines the assessment of the severity of lesions and the area affected into a single score in the range 0 (no disease) to 72 (maximal disease).

The body is divided into four sections head (H) (10% of a person's skin); arms (A) (20%); trunk (T) (30%); legs (L) (40%)). Each of these areas is scored by itself, and then the four scores are combined into the final PASI. For each section, the percent of area of skin involved, is estimated and then transformed into a grade from 0 to 6:
0% of involved area, grade: 0
<10% of involved area, grade: 1
10-29% of involved area, grade: 2
30-49% of involved area, grade: 3
50-69% of involved area, grade: 4
70-89% of involved area, grade: 5
90-100% of involved area, grade: 6

Within each area, the severity is estimated by three clinical signs: erythema (redness), induration (thickness) and desquamation (scaling). Severity parameters are measured on a scale of 0 to 4, from none to maximum. The sum of all three severity parameters is then calculated for each section of skin, multiplied by the area score for that area and multiplied by weight of respective section (0.1 for head, 0.2 for arms, 0.3 for body and 0.4 for legs).

PASI=0.1×(E_{H}+I_{H}+D_{H})×A_{H}+0.2×(E_{A}+I_{A}+D_{A})×A_{H}+0.3×(E_{T}+I_{T}+D_{T})×A_{T}+0.4×(E_{L}+I_{L}+D_{L})×A_{I}

Where E-Erythema, I-Induration and D is desquamation and (head (H) (10% of a person's skin); arms (A) (20%); trunk (T) (30%); legs (L) (40%))

Auspitz sign—It is the appearance of punctuate bleeding spots when psoriasis scales are scrapped off. Auspitz sign occurs because the capillaries under the epidermis are numerous and twisted, very close to the surface. Auspitz sign is used as a diagnostic tool for psoriasis. The combination of inflamed, thickened skin with silvery scale and Auspitz sign however appears to be unique to psoriasis. It is measured in the severity scale from 3 (severity), to 0 (Normal), with moderate (1).

In addition at each visit all the cases were evaluated for efficacy and any adverse effects, vital examination and laboratory investigation. The changes in various parameters from baseline values and the values until the and of the study were evaluated by “Paired ‘t’ Test”.

The demographic details of the subjects at entry are listed in table. All the 10 enrolled completed the study and the data were available for analysis. Treatment with antipsoriasis cream showed improvement in various clinical parameters of psoriasis which includes erythema, induration, dry silvery plaques and Auspitz sign and PASI score. Improvement was significant in clinical symptoms such as erythema, induration and Auspitz sign score. There was also a improvement in dry silvery plaques and PASI score.

There were no clinically significant adverse reactions, either reported or observed, during the entire study period and overall compliance to the treatment was excellent. Significant symptomatic relief was observed with Antipsoriasis cream in patients presenting with mild to moderate psoriasis. There were no clinically significant adverse reactions, either reported or observed, during the entire study period and overall compliance to the treatment was excellent. Thus it can be concluded that Antipsoriasis cream is effective and safe in patients suffering from mild and moderate psoriasis, without any observable adverse effects.

TABLE 5
Table: Demographic data of patients on entry (n = 10)

| Parameter | Antipsoriasis cream |
|---|---|
| Age in years (mean ± SD) | 26.18 ± 10.65 |
| Sex ratio/M:F | 04:06 |
| Diet (veg:mixed) | 07:03 |
| Duration of illness (months) | 15.36 ± 8.76 |

TABLE 6
Table: Effect of anti-psoriasis cream in clinical parameters of Anti-psoriasis cream

| | Anti-psoriasis cream (n = 10) | | | |
|---|---|---|---|---|
| Clinical | | End of 1^{st} | End of 2^{nd} | End of 3^{rd} |
| parameter score | At entry | month | month | month |
| Erythema score | 2.68 ± 1.48 | 2.04 ± .1.12 | 1.44 ± 0.52* | 0.8 ± 0.57** |
| Induration score | 2.34 ± 1.22 | 1.94 ± .1.33 | 1.28 ± 0.45* | 0.4 ± 0.37** |
| Dry silvery | 2.23 ± 1.66 | 2.14 ± .1.21 | 1.84 ± 0.42 | 1.04 ± 0.87 |
| plaques | | | | |
| (Desquamation) | | | | |
| Auspitz sign score | 2.13 ± 1.34 | 1.75 ± .1.3 | 0.86 ± 0.32* | 0.45 ± 0.27** |
| PASI score | 43.24 ± 10.38 | 32.67 ± 09.52 | 27.56 ± 10.72 | 19.23 ± 6.22 |

*p < 0.01 as compared to the at entry values
**p < 0.001 as compared to the at entry values

### Results:

From the results of the animal studies it was found that composite extract from the herbal blend AP (A) and Boswellia serrata extract (AS-BE-01) treated animals exhibited significant increase in percentage orthokeratosis, which was comparable to that of Tritenoin preparation.

In the follow up study, all the extracts showed increase trend of orthokeratosis but only APPC and APBN showed statistically significant increase. Calcipotriol (0.005%), even though showed increase in para to orthokeratosis conversion, the difference was not found to be statistically significant.

Although this invention has been described by example and with reference to possible embodiment thereof, it is to be understood that modifications or improvements may be made thereto without departing from the scope of the invention.

We claim:

1. A herbal composition having anti-psoriatic activity comprising effective amounts of extracts of Psoralia corylifolia, Luffa acutangula, Rubia cordifolia, Boswellia serrata, Brassica nigra, oils of Azadirachta indica, Linum usitatissimum, Vitis vinifera, Brassica nigra and suitable cosmeceutically acceptable carriers and additives wherein the said composition is in lotion or cream formulation.

2. The herbal composition as claimed in claim 1, wherein the extracts are obtained either by separate extraction or from the blend of Psoralia corylifolia, Luffa acutangula, Rubia cordifolia, Boswellia serrata, Brassica nigra, and oils of Azadirachta indica, Linum usitatissimum, Vitis vinifera, Brassica nigra parts.

3. The herbal composition as claimed in claim 1, wherein the percent weight by weight composition comprises 0.5-2% Psoralia corylifolia, 0.1-1% Luffa acutangula, 0.05-1% Rubia cordifolia, 0.05-1% Boswellia serrata, 1-3% Brassica nigra extract, 0.5-4% Azadirachta indica, 0.01-1% Linum usitatissimum, 0.2-2% Vitis vinifera, 0.1-2% Brassica nigra oil and cosmeceutically acceptable carriers.

4. The herbal composition as claimed in claim 1, wherein:
a. the extracts are obtained from the specific parts of the herbs preferably seeds of Psoralia corylifolia, seeds of Luffa acutangula, roots of Rubia cordifolia, gum resins of Boswellia serrata, seeds of Brassica nigra, and
b. the oils are obtained from the specific parts of herbs preferably seeds of Azadirachta indica, seeds of Linum usitatissimum, seeds of Vitis vinifera, seeds of Brassica nigra.

5. The herbal composition as claimed in claim 1 wherein the suitable cosmeceutical carrier is selected from the group of Xanthum gum, Aloe vera water, Glycerine, Carrageenan gum, Isopropyl Myristate, or any other carrier known in the art, or mixtures or combinations thereof.

6. The herbal composition as claimed in claim 1 wherein the additives are in the form of emulsifiers, preservatives, dispersants, solvents and catalytic enzyme.

7. The herbal composition as claimed in claim 6, wherein the said preservative is preferably sodium benzoate.

8. The herbal composition as claimed in claim 6, wherein the said catalytic enzyme is preferably Biovert Enzyme.

9. A herbal lotion having anti-psoriatic activity comprising of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 1.5-3% w/w alcoholic extract of Brassica nigra, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

10. A herbal lotion having anti-psoriatic activity comprising of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 0.5-2% w/w Psoralea corylifolia seed extract, 0.2-1% w/w Luffa acutangula seed extract, 0.05-0.8% w/w Rubia cordifolia root extract, 0.05-0.8% w/w Boswellia serrata gum resin extract, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

11. The herbal composition as claimed in claim 1, wherein the said extracts are obtained by at least one of the method selected from the group of percolation method, hot-soxlation method or super-critical-fluid extraction method.

12. The herbal composition as claimed in claim 1, wherein the said extracts are obtained by percolation method comprising the following steps of:
a. Shade drying the material;
b. Pulverizing the material obtained in step a. to coarse powder;
c. Placing the powdered material in different percolators and extracting with n-hexane, acetone, ethyl alcohol, methanol, ethyl alcohol and water (1:1), methanol and water (1:1) and water at room temperature for 24 h to 48 h.; and
d. Filtering the plant extracts and concentrating to dryness using a rotatory evaporator or on steam bath at optimum temperature under reduced pressure.

13. The herbal composition as claimed in claim 1, wherein the said extracts are obtained by hot-soxlation method comprising the steps of:
a. Placing the coarse powdered material in each soxlator using solvents n-hexane, acetone, ethyl alcohol, methanol, ethyl alcohol and water (1:1), methanol and water (1:1) and water at refluxing temperature of each solvent;
b. recycling the process until extraction is completed; and
c. filtering the plant extracts and concentrating to dryness using rotatory evaporator or on steam bath at optimum temperature.

14. The herbal composition as claimed in claim 1, wherein the said extracts are obtained by super-critical-fluid extraction method comprising the steps of:
a. Shade drying the material;
b. Pulverizing the dried material to coarse powder;
c. Placing the material in a SCF extractor at the temperature ranging from 40-50° C. at high pressure range of 300-350 bar using carbon dioxide as super critical fluid for extraction upto 4 to 6 hours; and
d. Collecting the extract in the collection vessel and evaporating at room temperature to remove any further residues of carbon dioxide.

15. A herbal lotion having anti-psoriatic activity comprising of 1-2% w/w Azadirachta indica seed oil, 0.1-1% w/w Linum usitatissimum seed oil, 0.5-1.25% w/w Vitis vinifera seed oil, 0.5-1.25% w/w Brassica nigra seed oil, 1.5-3% w/w herbal blend extract, 3-5% w/w sunflower oil, 3-5% w/w almond oil, 0.1-0.5% w/w Cocoa butter, 2-4% w/w Olivem 1000, 75-80% w/w Aloe vera water, 0.1-0.5% w/w Xanthum gum, 2-5% w/w Glycerine, 0.02-0.1% w/w Biovert Enzyme and 0.3-0.7% w/w sodium benzoate.

16. The herbal lotion as claimed in claim 15, wherein the herbal blend comprises of the seeds of Psoralia corylifoia, seeds of Luffa acutangula, roots of Rubia cordifolia and gum resins of Boswellia serrata in the ratio range of 45:20:15:20 to 50:25:10:15.

17. The herbal lotion as claimed in claim 15 wherein the said herbal blend is prepared by:
a. percolation method comprising the steps of:
i. Shade drying the material of herbal blend;
ii. Pulverizing the material obtained in step i. to coarse powder;
iii. Placing the powdered herbal blend in different percolators and extracting with n-hexane, acetone, ethyl alcohol, methanol, ethyl alcohol and water (1:1), methanol and water (1:1) and water at room temperature for 24 h to 48 h.; and
iv. Filtering the plant extracts and concentrating to dryness using a rotatory evaporator or on steam bath at optimum temperature under reduced pressure;
OR
b. hot-soxlation method comprising the steps of:
i. Placing the coarse powdered material of herbal blend in each soxlator using solvents n-hexane, acetone, ethyl alcohol, methanol, ethyl alcohol and water (1:1), methanol and water (1:1) and water at refluxing temperature of each solvent;
ii. recycling the process until extraction is completed; and
iii. filtering the plant extracts and concentrating to dryness using rotatory evaporator or on steam bath at optimum temperature.

18. A herbal cream having anti-psoriatic activity comprising of 3-5% w/w cetearyl Olivate and sorbitan olivate, 10-16% w/w sesame oil, 10-20% w/w Bees wax, 2-8% w/w isopropyl myristate, 0.1-1% w/w sorbic acid, 0.2-1.5% w/w Brassica nigra seed oil, 0.2-1.5% w/w Vitis vinifera seed oil, 0.1-1% w/w Linium usitatissimum seed oil, 0.5-2% w/w Pongamia seed oil, 5-15% w/w glycerine, 0.1-1% w/w Xanthum gum, 0.05-1% carrageenan gum, 1-5% w/w coco glucoside, 1-5% w/w Brassica nigra extract, 0.05-1% w/w sodium benzoate, 0.05-1% w/w potassium sorbate, 0.05-1% w/w Vitamin E acetate, 0.05-2% w/w perfume and 35-50% w/w purified water.

19. A method of preparation of a herbal lotion having anti-psoriatic activity comprising the following steps of:
a. Weighing the oil phase ingredients separately and heating the same in sterilized vessels up to a temperature ranging from 75-85° C.;
b. Dispersing xanthum gum in 10% Aloe vera water;
c. Adding glycerin-water and mixing the same with the alcoholic extract of Brassica nigra;
d. Heating the vessel containing water phase ingredients obtained in steps b. and c. to a temperature ranging from 75-85° C.;
e. Mixing the oil phase obtained in step a. with water phase obtained in step d. at temperature ranging from 75-85° C. and stirring the same properly; and
f. Mixing the preservatives in the phase obtained in step e. at temperature range less than 40° C. and stirring the same properly.

20. A method of preparation of a herbal cream having anti-psoriatic activity comprising the following steps of:
a. Heating Cetearyl Olivate & Sorbitan Olivate, sesame oil, Bees wax, isopropyl myristate, Sorbic acid and pongamia oil to a temperature ranging from 75-85° C. (Phase A);
b. Dispersing Xanthan gum and carrageenan gum in glycerine, adding this dispersion to purified water in main vessel, adding coco-glucoside in the main vessel, dissolving sodium benzoate and potassium sorbate in small quantity of water and adding the same to the main vessel, followed by adding Brassica nigra extract in the main vessel, heating the mixture thus obtained to a temperature ranging from 75-85° C. (Phase B);
c. Adding phase A to Phase B at a temperature ranging from 75-85° C. under homogenizer for 15-25 minutes;
d. Cooling the homogenized mixture obtained in step c. with normal water;
e. Adding Vitamin E acetate and perfume at a temperature ranging from 40-50° C.;
f. Mixing the content obtained in step e. for 10-20 minutes.
